(19) United States
(12) Patent Application Publication
(10) Pub. No.: US 2020/0009767 A1
(43) Pub. Date: Jan. 9, 2020

(54) Method and Device for Manufacturing Dissolving Microneedle

(71) Applicant: YOUWE (ZHUHAI) BIOTECHNOLOGY CO., LTD., Zhuhai, Guangdong (CN)

(72) Inventor: Chengguo LI, Zhuhai, Guangdong (CN)

(21) Appl. No.: 16/490,245

(22) PCT Filed: Feb. 27, 2018
(86) PCT No.: PCT/CN2018/077368

§ 371 Date: Aug. 30, 2019

(30) Foreign Application Priority Data
Mar. 2, 2017 (CN) 201710119814.0

(51) Int. Cl. B29C 41/10; A61K 9/00; A61M 37/00

(52) U.S. Cl. CPC B29C 41/10; A61K 9/0021; A61M 37/0015; B29K 2105/0058; A61M 2207/10; A61M 2037/0023; A61M 2037/0053

(57) ABSTRACT

A method and a device for manufacturing dissolving microneedle, wherein the method comprises manufacturing a mold with a plurality of microporous cavities, and manufacturing a polymer solution to be sprayed into the microporous cavity by a high pressure spraying device to enable the sprayed solution to fill in each microporous cavity. After the dryness of the solution, a microneedle is extracted from the mold. The process for manufacturing the microneedle is without vacuum pumping or any load of centrifugal force, and the process is simple and the excellent rate is high.

### CROSS-REFERENCE TO RELATED APPLICATIONS

This application claims the benefit of International Patent Application No. PCT/CN2018/077368 filed Feb. 27, 2018, which claims priority to Chinese Patent Application No. CN201710119814.0 filed with the China Patent Office on Mar. 2, 2017, entitled “Method for Manufacturing Dissolving Microneedle”, the entirety of which is incorporated herein by reference.

### TECHNICAL FIELD

The present application relates to the field of medical devices, and more particularly to a method and a device for manufacturing dissolving microneedle.

### BACKGROUND

A new type of painless transdermal drug delivery technique by means of dissolving microneedle drug delivery, which is able to create micrometer-scale drug delivery channels on the skin painlessly, and to enhance the skin's permeability to active substances or drugs, especially the permeability to macromolecular drugs. Due to the advantages of painlessness, safety and ease in operation, etc., the dissolving microneedle drug delivery technique is expected to be the direction of transdermal drug delivery to human body in the future.

The height of microneedle used in microneedle drug delivery technique, generally ranges from 50 to 1000 micrometers. The microneedle may penetrate the stratum corneum of the skin of which function as natural protection (usually only 10 to 20 micrometers), but may not reach the deep skin with abundant nerve endings, so the sense of pain never occur and is suitable to release drugs or active substances into blood or cells by means of transdermal injection of a small amount of active substances, drugs, genes, proteins or vaccines, etc.

Dissolving microneedle are made of biodegradable material as a substrate. Besides possessing the advantages of conventional microneedle, the biodegradable property enables the dissolving microneedle to solve the difficulty once the breakage of microneedle in the skin occurs. What's more, it also increases the drug loading and broadens the application scope of microneedle to some extent. Therefore, the biodegradable microneedle are expected to be an ideal carrier for transdermal drug delivery system and possess very bright application value.

A dissolving microneedle patch is a common method for the use of microneedle and many applicants have filed application for the microneedle patch. What's more, researchers in the University of Georgia of the United States proposed a method for manufacturing biodegradable polymer microneedle by etching glass or using photolithography to form a mold. In the article “Biodegradable polymer microneedle: Fabrication, mechanisms and transdermal drug delivery”, a method of manufacturing dissolving microneedle by micro mold was proposed. In this method, a silicon or high molecular polydimethylsiloxane mold that can be used for micro needle mold is produced by Micro Electro Mechanical Systems technology, then the biodegradable polymers mixed with active substances are dissolved (hyaluronic acid, methylolated cellulose, polyvinylpyrrolidone, etc.) or melted (polyglycolic acid, polylactic acid or polylactic acid-polyglycolic acid copolymer), and poured or filled into a silicon or polydimethylsiloxane microneedle mold, and the polymer solution is solidified into a microneedle shape by a process of high-temperature vacuum drying, finally the microneedle is manufactured by separating the solidified microneedle from the mold.

To refer to FIG. 1, when manufacturing a dissolving microneedle 13, firstly, the soluble polymer material to be made into the solid microneedle and the drug to be loaded needs to be made into a polymer solution, and then the polymer solution is injected into a microneedle mold 10. By centrifuging and vacuum pumping, the polymer solution is filled into a microporous cavity 11 of the microneedle mold 10. Generally, multiple times of centrifuging and vacuum pumping are required to allow the mixed solution to be fully filled into the microporous cavity 11 of the microneedle mold 10. Then, the polymer solution in the microneedle mold 10 is solidified, and finally the solidified microneedle 13 is separated from the microneedle mold 10 to form a dissolving microneedle 13.

Since the concave microporous cavity 11 of the microneedle mold 10 is at submicrometer level, the polymer solution with high viscosity 12 may not spontaneously fill the mixed solution into the microporous cavity 11 of the microneedle mold 10 due to the effect of the surface tension. The incomplete fill of the polymer solution 12 into the microporous cavity 11 of the microneedle mold 10 may lead to that the incomplete form of a needle head of microneedle 13 which finally formed, or the solidified microneedle 13 cannot be completely separated from the microneedle mold 10, etc.

In order to solve this problem, the microneedle is mainly produced by applying centrifugal force or vacuum pumping on the microneedle mold 10 to lead the polymer solution to be filled into the microporous cavity 11 of the microneedle mold 10. In the present microneedle manufacturing methods, the above process needs to be repeated for several times to ensure the polymer solution to be completely filled into the microporous cavity 11. The present microneedle manufacturing methods requiring repeated processing by means of centrifugal force or vacuum pumping applied to the microneedle mold 10, which leads to the instability of each batch during the production of microneedle and restricts its application in the industrialization of dissolving microneedle.

### SUMMARY

The present application provides a method and a device for manufacturing dissolving microneedle with simple process and high manufacturing efficiency.

The embodiments in the present application are realized as follows:

A method for manufacturing dissolving microneedle comprises manufacturing a microneedle mold with a plurality of microporous cavities, and spraying a polymer solution into the microporous cavity by a high pressure spraying device to ensure that the polymer solution filled in each of the microporous cavities, and extracting the solidified microneedle from the microneedle mold after the polymer solution is dried.

According to the foregoing solution, during manufacturing microneedle, the polymer solution with high viscosity is sprayed into the microporous cavity by a high pressure spraying device. The polymer solution with high viscosity is pushed into the microporous cavity by the high pressure, which is able to overcome the surface tension of the polymer solution, thereby ensures the polymer solution to be filled into the microporous cavity directly. Thus, the process of manufacturing microneedle is very simple, and it is unnecessary to fill the polymer solution into the microporous cavity by applying centrifugal force or vacuum pumping, etc. Therefore, the manufacturing process of the microneedle is simplified and the production efficiency of microneedle is improved, thereby the production cost of dissolving microneedle is reduced.

Optionally, spraying the polymer solution into the microporous cavity by the high pressure spray device comprises spraying the atomized polymer solution into said microporous cavity through a high pressure atomizing device.

As the particle size of the atomized polymer solution is very small and may be smoothly filled into the microporous cavity by the effect of high pressure, the manufacture of microneedle is very simple.

Optionally, the size of the atomized polymer solution sprayed by the high pressure atomizing device ranges from 0.1 to 100 micrometers.

Optionally, spraying the polymer solution into the microporous cavity through the high pressure spray device comprises spraying droplets of the polymer solution into the microporous cavity through a high pressure spotting device.

Since the droplets of the polymer solution are sprayed into the microporous cavity by a high-pressure spotting device, preferably, a spray nozzle of the spotting device is just right in front of each microporous cavity, to spray the droplets into the microporous cavity by high pressure spraying and the droplets may be smoothly filled into each microporous cavity.

Optionally, the size of the droplets of the polymer solution sprayed by the high pressure spotting device ranges from 0.1 to 1500 micrometers.

Optionally, the polymer solution is sprayed directly into the microporous cavity.

Optionally, before spraying said polymer solution into the microporous cavity, water or an active drug solution is sprayed into the microporous cavity, and the polymer solution is sprayed onto the water or the active drug solution, and the microneedle are extracted after the polymer solution is uniformly mixed with the water or the active drug solution and dried.

Thus, after the water or active drug is sprayed into the microporous cavity, the water or active drug may be more easily filled into the microporous cavity due to the low viscosity, and then sprayed the polymer solution with high viscosity. The polymer solution may be uniformly mixed into the water or active drug due to the property of intersolubility, thereby to achieve the purpose of filling the polymer solution in the microporous cavity.

Optionally, before the polymer solution is sprayed into the microporous cavity, a surfactant is sprayed to the polymer solution, and the polymer solution is sprayed onto the surfactant, and the microneedle are extracted after the polymer solution is uniformly mixed with the surfactant and dried.

Thus, the surfactant may be filled into the microporous cavity due to the low viscosity, in addition to the intersolubility of the polymer solution and the surfactant, the above factors enable the polymer solution to be filled into the microporous cavity after the polymer solution is uniformly mixed with the surfactant, and thereby the manufacturing process of microneedle is simplified.

Optionally, said surfactant is filled onto a surface of the microporous cavity, and there is a filling space formed in the microporous cavity that filled with said surfactant.

Optionally, said polymer solution is a polymer mixed solution mixed with an active drug; said polymer mixed solution is sprayed into the filling space by the high pressure spray device.

Thus, the polymer mixed solution mixed with the active drug is filled into the filling space formed by the surfactant in the microporous cavity, and when the surfactant is degraded, the active drug in the polymer mixed solution and the surfactant penetrate into the skin together.

Optionally, said high pressure spray device sprays a polymer solution into the microporous cavity at a pressure of less than 3.2 MPa.

Optionally, after the polymer solution is sprayed into the microneedle mold by the high pressure spray device, the polymer solution is filled into each of the microporous cavities, and after the dryness of polymer solution, the polymer solution merely adheres to the inner wall of the microporous cavity.

Optionally, the polymer solution is sprayed to the microneedle mold by the high pressure spray device and adheres to the surface of the microporous cavity of the microneedle mold, thereby to form a hollow dissolving microneedle; the drug droplets to be loaded are sprayed into microneedle with the hollow structure through a spray nozzle of a high pressure spotting device, and the drug droplets are filled into microneedle to form a conical structure.

Optionally, a sticky patch is pasted onto the surface of the microneedle, and then the sticky patch is in contact with the surface of the dissolving microneedle by pressing; the dissolving microneedle pasted on the sticky patch is separated from the microneedle mold by lifting and pulling.

Optionally, said polymer solution is a polymer solution mixed with a drug.

The device for manufacturing dissolving microneedle in the present application comprises a microneedle mold and a high pressure spray device. The microneedle mold has a plurality of microporous cavities and the high pressure spray device is configured to spray a polymer solution into the microporous cavity to make the polymer solution to be filled in each of the microporous cavities.

Compared to the prior art, the beneficial effects of the embodiments of the present application include, for example:

In summary, the method is simple in process, high in manufacturing efficiency and low in production cost.

### BRIEF DESCRIPTION OF THE DRAWINGS

In order to more clearly illustrate the technical solutions of the embodiments in the present application, the drawings to be used in the embodiments will be briefly described as follows. It should be understood that the following drawings only illustrate some embodiments of the present application and is not the limit of the scope, and other related drawings according to these drawings without any creative work can be obtained by the skilled in the art.

FIG. 1(a), FIG. 1(b), FIG. 1(c), and FIG. 1(d) is a schematic view of a present manufacturing process of dissolving microneedle;

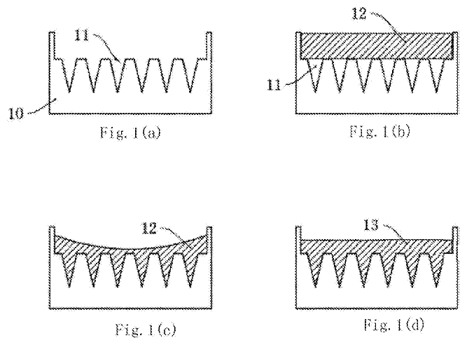

FIG. 2(a), FIG. 2(b), and FIG. 2(c), is a schematic view of the manufacturing process of the method for manufacturing dissolving microneedle in the first embodiment of the present application;

FIG. 3(a), FIG. 3(b), FIG. 3(c), and FIG. 3(d) is a schematic view of the manufacturing process of the method for manufacturing dissolving microneedle in the second embodiment of the present application;

FIG. 4(a), FIG. 4(b), FIG. 4(c), and FIG. 4(d) is a schematic view of the manufacturing process of the method for manufacturing dissolving microneedle in the third embodiment of the present application;

FIG. 5(a), FIG. 5(b), and FIG. 5(c) is a schematic view of the manufacturing process of the method for manufacturing dissolving microneedle in the fourth embodiment of the present application;

FIG. 6(a), FIG. 6(b), FIG. 6(c), and FIG. 6(d) is a schematic view of the manufacturing process of the method for manufacturing dissolving microneedle in the fifth embodiment of the present application;

FIG. 7(a), FIG. 7(b), FIG. 7(c), and FIG. 7(d) is a schematic view of the manufacturing process of the method for manufacturing dissolving microneedle in the sixth embodiment of the present application;

FIG. 8(a), FIG. 8(b), FIG. 8(c), and FIG. 8(d) is a schematic view of an extraction process of the method for manufacturing dissolving microneedle in the first embodiment of the present application;

FIG. 9(a), FIG. 9(b), FIG. 9(c), and FIG. 9(d) is a schematic view of an extraction process of the method for manufacturing dissolving microneedle in the fourth embodiment of the present application;

FIG. 10(a), FIG. 10(b), FIG. 10(c), FIG. 10(d), and FIG. 10(e) is a schematic view of an extraction process of the method for manufacturing dissolving microneedle in the seventh embodiment of the present application;

FIG. 11(a), FIG. 11(b), FIG. 11(c), FIG. 11(d), and FIG. 11(e) is a schematic view of an extraction process of the method for manufacturing dissolving microneedle in the eighth embodiment of the present application;

FIG. 12(a), FIG. 12(b), FIG. 12(c), and FIG. 12(d) is a schematic view of the manufacturing process of the method for manufacturing dissolving microneedle in the ninth embodiment of the present application;

FIG. 13(a), FIG. 13(b), and FIG. 13(c) is a schematic view of the use of the method for manufacturing dissolving microneedle in the first embodiment of the present application;

FIG. 14(a), FIG. 14(b), and FIG. 14(c) is a schematic view of the use of the method for manufacturing dissolving microneedle in the seventh embodiment of the present application;

FIG. 15(a) and FIG. 15(b) is a schematic view of the use of the method for manufacturing dissolving microneedle in the ninth embodiment of the present application;

FIG. 16(a) and FIG. 16(b) is a schematic view of the use of the method for manufacturing dissolving microneedle in the tenth embodiment of the present application.

Notes: 10—microneedle mold; 11—microporous cavity; 12—polymer solution; 13—microneedle; 5—skin; 20—microneedle mold; 21—microporous cavity; 22—polymer solution; 23—microneedle; 25—high pressure atomizing nozzle; 27—sticky patch; 28—pressing block; 29—active drug; 30—microneedle mold; 31—microporous cavity; 32—water; 33—polymer solution; 34—polymer mixed solution; 35—high pressure atomizing nozzle; 40—microneedle mold; 41—microporous cavity; 42—surfactant; 43—polymer solution; 44—polymer mixed solution; 45—high pressure atomizing nozzle; 47—filling space; 50—microneedle mold; 51—microporous cavity; 53—microneedle; 55—high pressure spray nozzle; 56—droplet; 57—sticky patch; 58—pressing block; 60—microneedle mold; 61—microporous cavity; 62—active drug solution; 64—polymer mixed solution; 65—spray nozzle; 66—droplet; 67—droplet; 70—microneedle mold; 71—microporous cavity; 72—surfactant; 73—filling space; 74—polymer mixed solution; 75—spray nozzle; 77—droplet; 80—microneedle mold; 81—microporous cavity; 82—polymer solution; 83—microneedle; 85—sticky patch; 86—pressing block; 89—active drug; 90—microneedle mold; 91—microporous cavity; 92—polymer solution; 93—microneedle; 95—sticky patch; 96—pressing block; 100—microneedle mold; 101—microporous cavity; 102—polymer solution; 103—conical structure; 105—spray nozzle; 106—drug droplet; 108—sticky patch; 109—pressing block; 110—drug-loaded portion; 112—microneedle shell; 122—microneedle.

### DETAILED DESCRIPTION

In order to illustrate the object, technical solutions and advantages of the embodiments of the present application more clearly, the technical solutions in the embodiments of the present application are described in a clear and complete manner with reference to the accompanying drawings. Apparently, embodiments described herein are a part of rather than all of the embodiments. The components of the embodiments of the present application, which are generally described and illustrated in the figures herein, may be arranged and designed in various different configurations.

Thus, the detailed description of the embodiments in the present application, which is set forth in the accompanying drawings, is not intended to limit the scope of protection of the present application, but is merely an illustration of the selected embodiments in the present application. All other embodiments obtained by those skilled in the art without creative work based on the embodiments of the present application shall fall into the scope of protection of the present application.

It should be noted that similar reference numerals and letters indicate similar terms in the following drawings, therefore, it is unnecessary to further define and explain the term in the subsequent drawings once it is defined in a drawing.

In the description of the present application, it should be noted that the orientation or positional relationship indicted by the terms “upper”, “lower”, “in/inside”, “out/outside”, etc. is based on the orientation or positional relationship shown in the drawings, or the orientation or positional relationship when the product is conventionally placed during use. It is only for the convenience to describe the application and simplification of the description, rather than indicating or implying that the device or component referred to herein should have a specific orientation, or should be constructed and operated in a specific orientation, thus it cannot be understood as the limit of the application. Moreover, the terms “first”, “second” and “third”, etc. are used only to distinguish the description, and cannot be understood as indicating or implying a relative importance.

In the description of the present application, unless otherwise specified and defined, the term “connection/connecting/connected” should be understood in a broad sense, for example, it may be a fixed connection, a detachable connection, or an integral connection; it may be a mechanical connection or an electrical connection; it may be a direct connection or an indirect connection through an intermediate medium, or an internal communication between two elements. The specific meaning of the above terms in the present application can be understood by those skilled in the art.

In the present application, the method for manufacturing dissolving microneedle is to spray a polymer solution into a microneedle mold by a high pressure spray device, for example, by means of spraying a polymer solution into a microneedle mold after atomization through a high pressure atomizing device, or spotting droplets of the polymer solution into a microporous cavity of a microneedle mold under high pressure in order to spray the polymer solution with high viscosity into the microneedle mold. The manufacturing efficiency of microneedle is enhanced and the microneedle production cost is reduced since it is unnecessary for the method in the present application to fill the polymer solution into the microporous cavity of the microneedle mold by vacuum pumping or applying a centrifugal force.

### First Embodiment

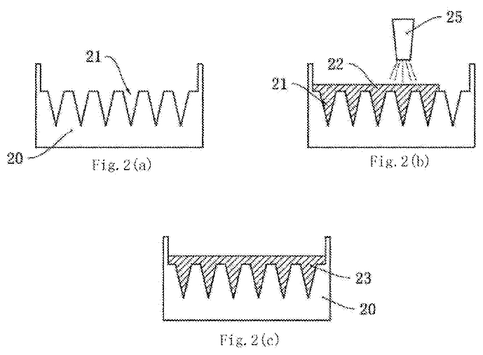

To refer to FIG. 2, a microneedle mold 20 was firstly manufactured in the microneedle manufacturing method of the present embodiment. The microneedle mold 20 had a plurality of microporous cavities 21, and the shape of each microporous cavity 21 was inverted conical. Of course, the microporous cavity may be other shapes and was not limited to the inverted conical. Preferably, the height of each microporous cavity 21 ranged from 50 micrometers to 1000 micrometers, and the inner diameter of the entrance of each microporous cavity 21 was less than 1000 micrometers.

The microneedle mold 20 may be prepared by a wet etch process, for example, arranging conical silicon microneedle in a 10×10 array, the length of array was 200 micrometers and a base diameter was 100 micrometers, or arranging conical silicon microneedle in a 20×20 array, the length of array was 800 micrometers and a base diameter was 300 micrometers, the microneedle were used as an inverted mold template to manufacture a microporous cavity in a polydimethylsiloxane (PDMS) mold.

After the manufacture of microneedle mold 20, the polymer solution was sprayed into the microneedle mold 20 by a high pressure atomizing device. For example, there was a high pressure atomizing nozzle 25 on the high pressure atomizing device, and the polymer solution was sprayed to the microneedle mold 20 from the high pressure atomizing nozzle 25. Specifically, as shown in FIG. 2, the polymer solution 22 was sprayed into each of the microporous cavities 21 and was filled in each microporous cavity 21.

Preferably, the pressure value of the high pressure atomizing nozzle 25 on the high pressure atomizing device in the embodiment applies to the polymer solution 22 ranged from 0 to 3.2 MPa, and the particle size of the polymer solution sprayed through the high pressure atomizing nozzle 25 was greater than 0.1 micrometers and far less than 100 micrometers, such as 10 micrometers or less. Preferably, the particle size of atomized polymer solution should be smaller than the inner diameter of the entrance of the microporous cavity 21 for the purpose that the polymer solution 22 would smoothly flow into the microporous cavity 21 more easily. Of course, the particle size of the atomized polymer solution 22 could be adjusted by adjusting the diameter of the orifice of the high pressure atomizing nozzle 25 on the high pressure atomizing device. Thus, to adjust the diameter of the orifice of the high pressure atomizing nozzle 25 according to the inner diameter of the entrance of the microporous cavity 21 of the microneedle mold 20, was for the purpose that the particle size of the atomized polymer solution 22 is adjustable.

As shown in FIG. 2, when the polymer solution was sprayed into all the microporous cavities 21 of the microneedle mold 20 by the high pressure atomizing nozzle 25, the polymer solution 22 was filled into each microporous cavity 21, and a microneedle 23 was formed through the solidification and separation of polymer solution 22.

The microneedle manufactured by the method in the present application are dissolving microneedle, which means the microneedle is able to dissolve in the human body. Therefore, the biodegradable substances may be used as the materials of polymer solutions. The used materials may include polyester, polyhydroxyalkanoate (PHA), poly(α-hydroxy acid), poly(β-hydroxy acid), poly(3-hydroxybutyrate-co-valerate) (PHBV), poly(3-hydroxypropionate) (PHP), poly(3-hydroxyhexanoate)(PHH), poly(4-hydroxy acid), polyphosphagens, PHA-PEG (polyhydroxyalkanoate-polyethylene glycol), ethylene vinyl alcohol copolymer (EVOH), ABS [poly(acrylonitrile, butadiene, styrene)] resin, 10-ethylene-vinyl acetate copolymer, poly(4-hydroxybutyrate), poly(4-hydroxyvalerate), poly(4-hydroxyhexanoate), poly(esteramide), polycaprolactone, polylactide, polyglycolide, poly(lactide-co-glycolide)(PLGA), polydioxanone, polyorthoester, polyether ester, polyanhydride, poly(glycolic acid-co-trimethylene carbonate), polyphosphate, polyphosphate carbamate, poly(amino acid)), polycyanoacrylate, poly(trimethylene carbonate), poly(iminocarbonate), poly(tyrosine carbonate), polycarbonate, poly(tyrosine aryl ester), polyalkylene oxalate, polyurethane, silicone, polyester, polyolefin, copolymer of poly-5-isobutylene and ethylene-α-olefin, styrene-isobutylene-styrene triblock copolymer, acrylic polymer and copolymer, ethylene halide polymer and copolymer, polyvinyl chloride, polyvinyl ether, polyvinyl methyl ether, poly(vinylidene chloride), poly(vinylidene fluoride), poly(vinylidene chloride), polyfluoroolefin, polyperfluoroolefin, polyacrylonitrile, poly vinyl ketone, polyvinyl aromatic compound, polystyrene, polyvinyl ester, polyvinyl acetate, ethylene-methyl methacrylate copolymer, acrylonitrile-styrene copolymer, polyamide, alkyd resin, poly oxidized methylene, polyimide, polyether, polyacrylate, polymethacrylate, polyacrylic acid-co-maleic acid, chitosan, dextran, cellulose, heparin, hyaluronic acid, alginate ester, inulin, starch or glycogen.

Preferably, the polymer solution used in the present embodiment is polyester, PHA, PHBV, PHP, PHH, PHA-PEG, poly(4-hydroxy acid), poly(α-hydroxy acid), poly(β-hydroxy acid), poly(4-hydroxybutyrate), poly(4-hydroxyvalerate), poly(4-hydroxyhexanoate), poly(esteramide), polycaprolactone, polylactide, polyglycolide, PLGA, 15-polydioxanone, polyorthoester, polyether ester, polyanhydride, poly(glycolic acid-co-trimethylene carbonate), polyphosphate, polyphosphate urethane, poly (amino acid), polycyanoacrylate, poly(trimethylene carbonate), poly(iminocarbonate), poly(tyrosine carbonate), polycarbonate, poly(tyrosine aryl ester), polyalkylene oxalate, polyphosphate creatine, chitosan, dextran, cellulose, heparin, hyaluronic acid, alginic acid, inulin, starch or glycogen. Of course, the above materials may be used either alone or in combination.

### Second Embodiment

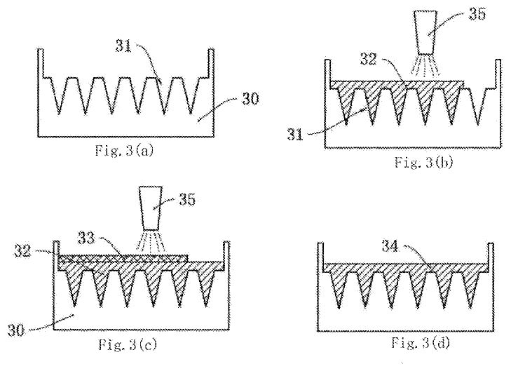

To refer to FIG. 3, to manufacture the microneedle according to the embodiment, firstly manufactured a microneedle mold 30, and there were a plurality of microporous cavities 31 in the microneedle mold 30. After the manufacture of the microneedle mold 30, water or active drug solution was sprayed into the microneedle mold 30, e.g. water or active drug solution was sprayed into the microneedle mold 30 by a high pressure atomizing nozzle 35 on a high pressure atomizing device. For example, in the present embodiment, water 32 was sprayed into a microneedle mold by the high pressure atomizing nozzle 35. It was easy to flow into the microporous cavity 31 for the water due to its low viscosity and small surface tension.

Then, the polymer solution 33 was sprayed to the microneedle mold 30 by the high pressure atomizing device. The polymer solution 33 was actually sprayed onto the water 32 as the microporous cavity 31 was already filled with water. Furthermore, as the polymer solution 33 may be mutually mixed with the water 32, e.g. the polymer solution 33 and water had good intersolubility, the polymer solution 33 and water 32 may be mixed in the microneedle mold 30 to form a polymer mixed solution 34. Thus, the polymer mixed solution 34 was actually filled into each of the microporous cavities 31. As shown in FIG. 3, the polymer mixed solution 34 formed by the uniformly mixed of the water 32 and the polymer solution 33 filled in each microporous cavity 31, thereby to form a microneedle with a solid structure.

### Third Embodiment

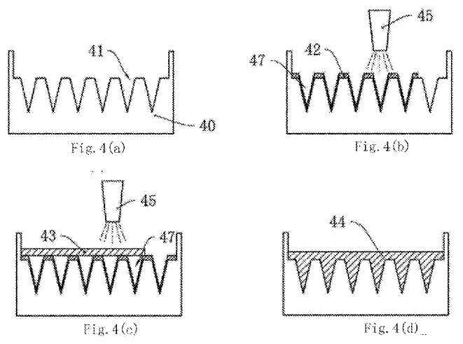

As shown in FIG. 4, firstly to manufacture a microneedle mold 40, and there were a plurality of microporous cavities 41 in the microneedle mold 40. Then the surfactant 42 was sprayed into the microporous cavity 41 by the high pressure atomizing device. As the low viscosity of the surfactant 42 in the embodiment and the small amount of surfactant sprayed into the microneedle mold 40, the surfactant 42 merely adhered to the inner wall of the microporous cavity 41 and was unable to fill in each microporous cavity 41, which led to the formation of a filling space 47 within the surfactant 42.

Then the atomized polymer solution 43 was sprayed into the microneedle mold 40 by the high pressure atomizing nozzle 45 on the high pressure atomizing device. As the surfactant 42 was hydrophilic and the polymer solution 43 was mutually soluble with the surfactant, the polymer solution 43 may be uniformly mixed with the surfactant 42 and filled in each microporous cavity 41 to form a polymer mixed solution 44, after the dryness of polymer mixed solution 44, a dissolving microneedle was formed. In the embodiment, the polymer mixed solution 44 may also be mixed with an active drug which was directly sprayed into the filling space 47 by a high pressure spray device.

### Fourth Embodiment

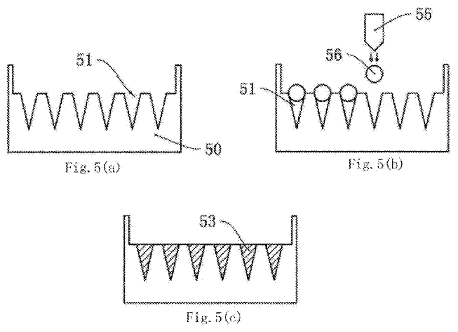

To refer to FIG. 5, droplets 56 of the polymer solution were dripped into the microneedle mold 50 by high pressure spotting in the present embodiment. Preferably, the height of each microporous cavity 21 ranged from 50 micrometers to 1000 micrometers, and the inner diameter of the entrance of each microporous cavity 21 was less than 1000 micrometers.

During manufacturing the microneedle, firstly a microneedle mold 50 was manufactured, and the microneedle mold 50 had a plurality of conical microporous cavities 51. After the microneedle mold 50 was manufactured, droplets 56 of the polymer solution were dripped into each microporous cavity 51 of the microneedle mold 50 by the high pressure spotting device. For example, a high pressure spray nozzle 55 of a high pressure spotting device was moved directly above a microporous cavity 51, then the droplets 56 of the polymer solution were dripped into the microporous cavity 51. Preferably, the pressure value of the high pressure spray nozzle 55 on the high pressure spotting device used in the present embodiment ranged from 0 to 3.2 MPa, and the particle size of the dripping droplets 56 of the polymer solution ranged from 0.1 to 1500 micrometers. As shown in FIG. 5, although the particle size of the droplet 56 was slightly larger than the inner diameter of the entrance of the microporous cavity 51, as the droplets 56 were dripped by the high pressure spraying, therefore the polymer solution overcame the surface tension of the polymer solution droplet 56 by the effect of the pressure and filled into each microporous cavity 51. After the dryness and solidification of the polymer solution, the dissolving microneedle 53 was formed.

### Fifth Embodiment

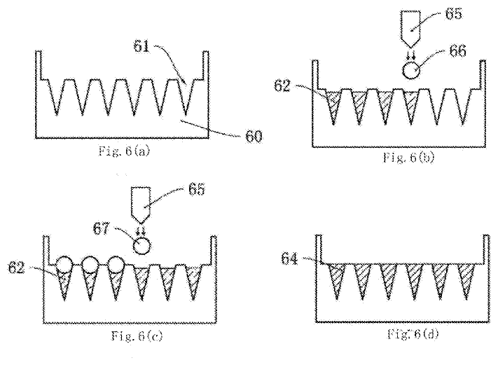

To refer to FIG. 6, to manufacture the microneedle according to the embodiment, firstly manufactured a microneedle mold 60, and the microneedle mold 60 had a plurality of microporous cavities 61. After manufacturing the microneedle mold 60, water or active drug solution was preliminarily sprayed into the microneedle mold 60. In the present embodiment, the active drug was sprayed into the microneedle mold 60 by a high pressure spotting device. For example, a droplet 66 of active drug was dripped to each microporous cavity 61 of the microneedle mold 60 by the spray nozzle 65 on a high pressure spotting device to form an active drug solution 62 in each microporous cavity 61. It was easy to flow into the microporous cavity 61 for the water due to its low viscosity and small surface tension.

Then, the polymer solution was sprayed to the microneedle mold 60 by a high pressure spotting device, for example, the droplets 67 of the polymer solution were dripped into each microporous cavity 61 by a high pressure spray nozzle 65. As the microporous cavity 61 was already filled with the active drug solution 62, the polymer solution was actually sprayed onto the active drug solution. Furthermore, as the polymer solution may be mutually mixed with the active drug solution 62, e.g. the polymer solution and the active drug solution 62 had good intersolubility, and the polymer solution may be mixed in the microneedle mold 60 to form a polymer mixed solution 64.

Thus, the polymer mixed solution 64 was actually filled into each microporous cavity 61 in the present embodiment. As shown in FIG. 6, the polymer mixed solution 64 formed by the uniformly mixed of the active drug solution 62 and the polymer solution filled in each microporous cavity 61, thereby to form a microneedle with a solid structure.

### Sixth Embodiment

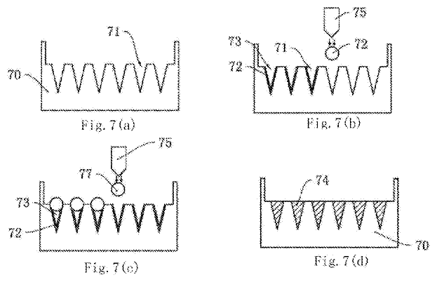

To refer to FIG. 7, in the present embodiment, firstly to manufacture a microneedle mold 70. The microneedle mold 70 had a plurality of microporous cavities 71. Then a surfactant 72 was sprayed into the microporous cavity 71 by a high pressure spotting device. For example, the droplets of the surfactant 72 were dripped into each microporous cavity 71 by the spray nozzle 75. As low viscosity of the surfactant 72 in the embodiment and the small amount of surfactant sprayed into the microneedle mold 70, the surfactant 72 merely adhered to the inner wall of the microporous cavity 71 and was unable to fill in each microporous cavity 71, which led to the formation of a filling space 73 within the surfactant 72.

Then the droplets 77 of the polymer solution were sprayed into the microneedle mold 70 by a spray nozzle 75 of a high pressure spotting device. As the filling space 73 was formed inside the surfactant 72, the droplets 77 actually dripped into the filling space 73. As the surfactant 72 was hydrophilic and the polymer solution was mutually soluble with the surfactant 72, the polymer solution may be uniformly mixed with the surfactant 72 and filled in each microporous cavity 71 to form a polymer mixed solution 74, after the dryness of polymer mixed solution, a dissolving microneedle was formed.

In the present embodiment, the polymer mixed solution 74 may also be mixed with an active drug which was directly sprayed into the filling space 73 by the high pressure spray device.

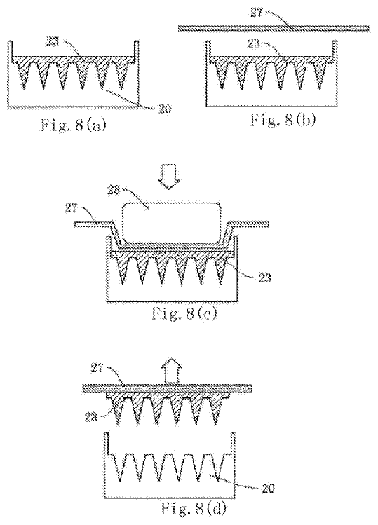

The following part may combine figures from FIGS. 8 to 11 to describe how to extract microneedle from a microneedle mold in different embodiments.

To Refer to FIG. 8, for example, in the first embodiment, a dissolving microneedle 23 was formed after the dryness of polymer solution in the microneedle mold 20. During extracting the microneedle 23, firstly a sticky patch 27 was covered onto the surface of the microneedle mold 20 where the microneedle 23 was formed. Then, the sticky patch 27 was in contact with and adhered to the surface of the dissolving microneedle 23 by pressing. One way was that a pressing block 28 was pressed on the sticky patch 27, to enable the sticky patch 27 to adhere to the surface of the microneedle 23. Finally, the dissolving microneedle 23 adhered to the sticky patch 27 was separated from the microneedle mold 20 by lifting and pulling, at this time, the microneedle 23 was attached to the sticky patch 27, so that the microneedle 23 could be extracted.

FIG. 8 showed a method for extracting a solid microneedle with a substrate, and for a microneedle without a substrate, the microneedle may also be extracted by the same way.

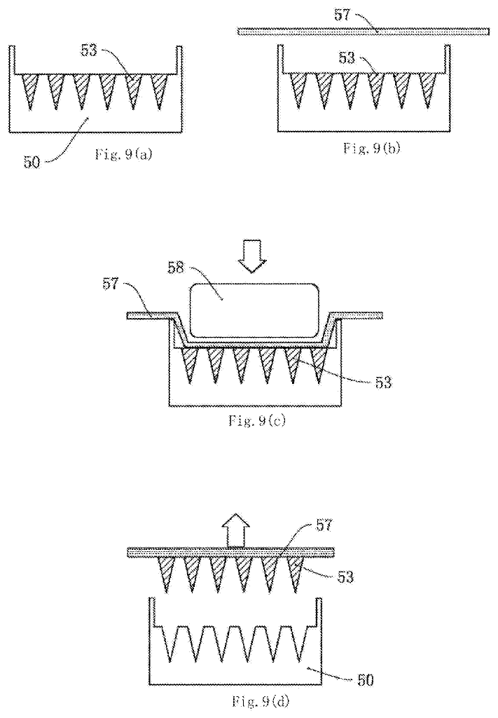

To refer to FIG. 9, the microneedle manufactured by the method described in the fourth embodiment was a solid microneedle 53 without a substrate. In the course of extracting this kind of microneedle 53, the sticky patch 57 was firstly pasted on the surface of the microneedle 53, and then the sticky patch 57 was in contact with and adhered to the surface of the dissolving microneedle 53 by pressing. One way was that a pressing block 58 was pressed on the sticky patch 57, so that the sticky patch 57 may adhere to the surface of the microneedle 53. Finally, the dissolving microneedle 53 adhered to the sticky patch 57 was separated from the microneedle mold 50 by lifting and pulling, at this time, the microneedle 53 was attached to the sticky patch 57, so that the microneedle 53 could be extracted.

### Seventh Embodiment

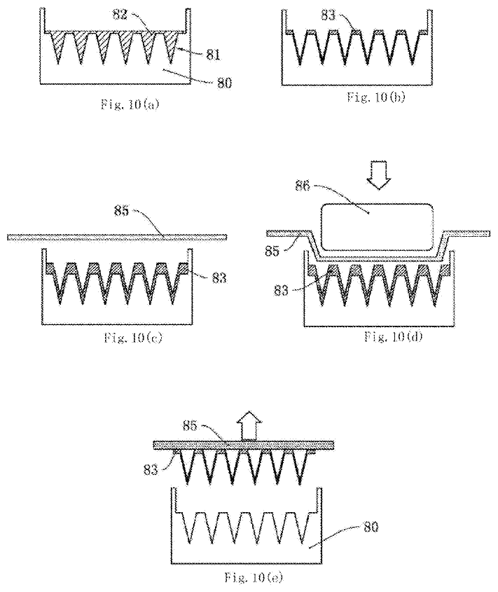

Of course, the present application may also manufacture a dissolving microneedle with a hollow structure. As shown in FIG. 10, after the manufacture of the microneedle mold 80, the polymer solution 82 with low concentration was sprayed into the microneedle mold 80 by a high pressure spray device, and then the polymer solution 82 with low concentration filled in each microporous cavity 81. After dryness, the polymer solution 82 merely adhered to the inner wall of the microporous cavity 81, thereby the dissolving microneedle 83 with a hollow structure was formed.

During extracting the microneedle 83, the sticky patch 85 was firstly pasted on the surface of the microneedle 83, and then the sticky patch 85 was in contact with and adhered to the surface of the dissolving microneedle 83 by pressing. One way was that a pressing block 86 was pressed on the sticky patch 85, so that the sticky patch 85 may adhere to the surface of the microneedle 83. Finally, the dissolving microneedle 83 adhered to the sticky patch 85 was separated from the microneedle mold 80 by lifting and pulling, at this time, the microneedle 83 was attached to the sticky patch 85, so that the microneedle 83 could be extracted from the microneedle mold 80.

### Eighth Embodiment

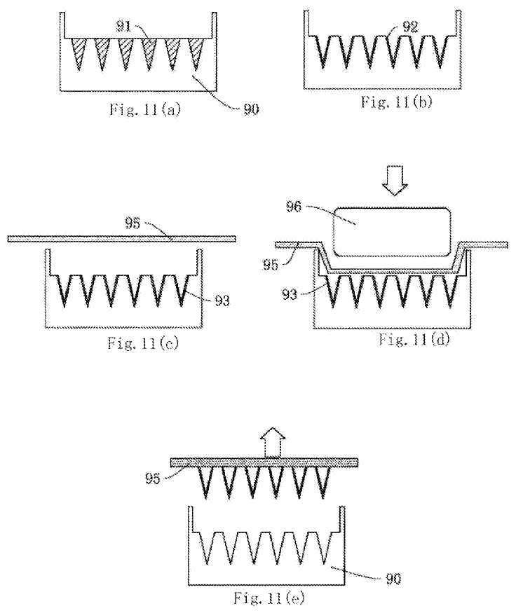

Of course, the present application may also manufacture the hollow dissolving microneedle without a substrate structure. As shown in FIG. 11, after the manufacture of the microneedle mold 90, the polymer solution 92 with low concentration was sprayed into the microneedle mold 90 by the high pressure spray device, and then the polymer solution 92 with low concentration filled in each microporous cavity 91. After dryness, the polymer solution 92 merely adhered to the inner wall of the microporous cavity 91, thereby the hollow dissolving microneedle 93 without a substrate structure was formed.

During extracting the microneedle 93, the sticky patch 95 was firstly pasted onto the surface of the microneedle 93, and then the sticky patch 95 was in contact with and adhered to the surface of the dissolving microneedle 93 by pressing. One way was that a pressing block 96 was pressed on the sticky patch 95, so that the sticky patch 95 may adhere to the surface of the microneedle 93. Finally, the dissolving microneedle 93 adhered to the sticky patch 95 was separated from the microneedle mold 90 by lifting and pulling, at this time, the microneedle 93 was attached to the sticky patch 95, so that the microneedle 93 may be extracted from the microneedle mold 90.

### Ninth Embodiment

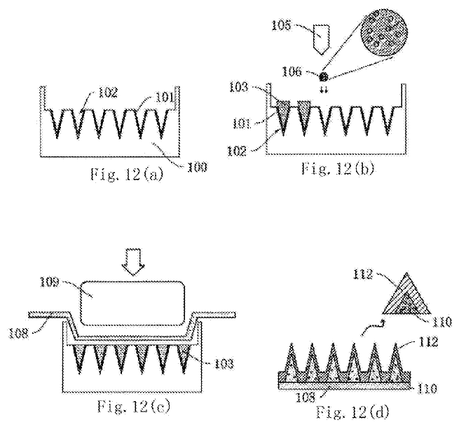

The method of the present application may also manufacture a capsule type microneedle. As shown in FIG. 12, firstly a polymer solution 102 with low concentration was sprayed into the microneedle mold 100 by high-pressure spraying, and the polymer solution 102 adhered to the surface of the microporous cavity 101 of the microneedle mold 100, thereby manufactured the microneedle with a hollow dissolving microneedle.

Then, the drug droplets 106 to be loaded were spotted into a microneedle with a hollow structure by high-pressure spotting, for example, by a spray nozzle 105 on a high pressure spotting device, and the drug droplets 106 were filled into the microneedle to form a conical structure 103.

During extracting the microneedle, after the drug droplet 106 is dripped onto the microneedle, a sticky patch 108 should be covered onto the microneedle mold 100 immediately, and the sticky patch 108 was in contact with and adhered to the dissolving microneedle by pressing. For example, by pressing the sticky patch 108 with a pressing block 109 and then the dissolving microneedle adhered to the sticky patch 108 was separated from the microneedle mold 100 by lifting and pulling, to form a “capsule type” drug-loaded dissolving microneedle patch. The microneedle patch comprised a sticky patch 108, a microneedle shell 112 and a drug-loaded portion 110 formed inside the microneedle.

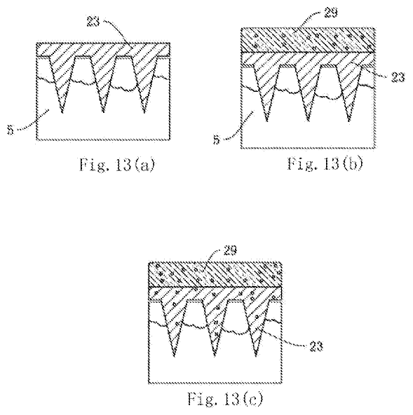

The following part combined figures from FIGS. 13 to 16 to describe the application methods of several dissolving microneedle patches. To refer to FIG. 13, a non-drug-loaded, solid dissolving microneedle 23 was manufactured in the first embodiment. The microneedle 23 was adhered by a sticky patch to form a dissolving microneedle patch. During the use of the patch, after penetrating the stratum corneum of the skin 5, the microneedle 23 was dissolved by contacting with water inside the skin 5 to form a transdermal drug delivery channel. At this time, active drug or functional cosmetics may be applied on the skin. As the microneedle patch was attached to the skin 5, a part of the active drug 29 or functional cosmetics was applied to the surface of the microneedle 23, and the active drug 29 or functional cosmetics applied onto the skin surface would be absorbed efficiently by the skin through microporous channels formed on the surface of the skin by microneedle.

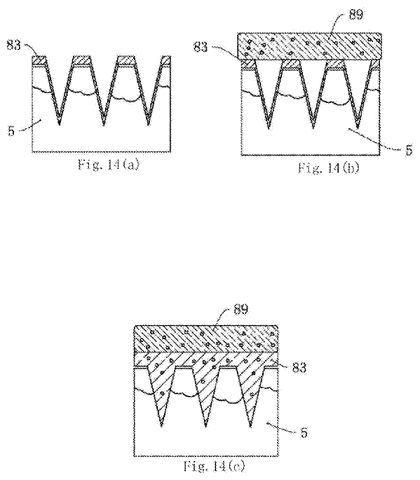

To refer to FIG. 14, the seventh embodiment provided a non-drug-loaded, hollow dissolving microneedle 83. The microneedle 83 was pasted by a sticky patch to form a microneedle patch, which may be directly applied to human skin, a transdermal drug delivery channel through the transmission of skin 5 was formed after the hollow dissolving microneedle 83 penetrating the stratum corneum of the skin 5. The active drug 89 or functional cosmetic applied to the skin surface and enter the hollow structure of the microneedle 83 may be dissolved in the skin 5 together with the microneedle 83, and absorbed by the skin efficiently.

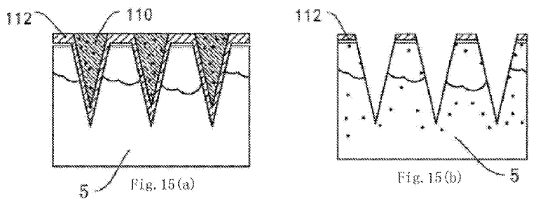

The ninth embodiment provided a “capsule type” drug-loaded microneedle. As shown in FIG. 15, the microneedle was pasted by a sticky patch to form a microneedle patch, which could be directly applied to human skin. After the microneedle shell 112 and the drug-loaded portion 110 penetrating the stratum corneum of the skin 5, the microneedle may be dissolved by contacting with the water in the skin, and the drug loaded by the microneedle would be dissolved into the skin 5 simultaneously, to achieve the goal of efficient absorption.

### Tenth Embodiment

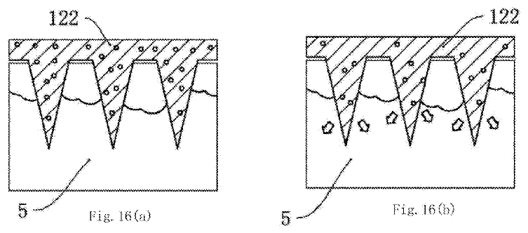

As shown in FIG. 16, the microneedle of the present embodiment was a drug-loaded, solid dissolving microneedle 122. The microneedle 122 was formed by spraying the polymer solution containing drug to the microneedle mold under the condition of high pressure and dryness. During extracting, the microneedle 122 was pasted by a sticky patch to form a microneedle patch. The microneedle patch may be directly applied to human skin 5.

During the use of microneedle patch, after the microneedle 122 penetrating the stratum corneum of the skin 5, the microneedle 122 was dissolved by contacting with water in the skin 5, and the drug loaded by the microneedle 122 would be dissolved into the skin simultaneously, to achieve the goal of efficient absorption.

From the above description, the main application of the dissolving microneedle patch of the present application is transdermal drug delivery. Therefore, during the manufacturing process of dissolving microneedle, the drug may be mixed with a soluble polymer solution to manufacture a dissolving microneedle. Drugs used in the present application are not particularly limited, for example, the drugs may include chemical drugs, protein drugs, peptide drugs, nucleic acid molecules and nanoparticles for gene therapy, etc. Drugs that could be used in the present application may include anti-inflammatory agents, analgesics, anti-arthritis agents, anti-caries agents, antidepressants, antipsychotics, neuroleptics, anxiolytics, anesthetic antagonists, anti-Parkinson's disease medicines, cholinergic agonists, anticancer agents, antiangiogenic agents, immunosuppressants, antiviral agents, antibiotic agents, appetite reducing agents, analgesic agents, anticholinergic agents, antihistamines, anti-migraine agents, hormonal drugs, coronary vasodilators, cerebral vasodilators or peripheral vasodilators, contraceptives, antithrombotic agents, diuretics, antihypertensive agents, cardiovascular disease and therapeutic agents, but are not limited to them herein.

Cosmetic functional ingredients loaded by the dissolving microneedle may include humectant such as glycerin, filaggrin, or a substance with a wrinkle-removing function such as hyaluronic acid (non-crosslinking, cross-linking), or a whitening agent such as hydroquinone, arbutin, aspergillic acid, glycolic acid or niacinamide, etc, or a skin rejuvenating substance, such as vitamin C, vitamin A, vitamin E, etc, or an acne-removing drugs, such as Madecassoside, Epigallocatechin Gallate(-), Salicylates, 2-hydroxybenzoic, Salicylic Acid, Beta Hydroxy Acid, etc.

A typical method for manufacturing microneedle in the present application was to manufacture the microneedle without hollow structure by use of 50% aqueous solution of hyaluronic acid (HA) (by weight or volume ratio). When needed, 1 mg/mL of methylene blue dye was added to achieve visualization experiments, or 20% aqueous solution of hyaluronic acid (HA) (by weight or volume ratio) was used to manufacture a microneedle with a hollow structure. When needed, 1 mg/mL of Congo red dye was added to achieve visualization experiments.

Then a hyaluronic acid solution was sprayed directly into the microporous cavity by a spray nozzle and compressed air. After the hyaluronic acid solution was sprayed into the microneedle mold, the microneedle was dried by a desiccant at room temperature (25° C.) for 10 minutes to 60 minutes, then transferred to a medical grade tape, the microneedle array was stored in a dry environment.

Alternatively, water or a drug solution was sprayed into the microporous cavity by a spray nozzle and compressed air, and then a hyaluronic acid solution was covered onto the surface of the microneedle mold that filled with the water or drug solution through a dispenser connected with a syringe pump at a speed of 1 μL/min to 3 μl/min. After the coverage of the hyaluronic acid solution on the mold, the microneedle was dried by the desiccant at room temperature (25° C.) for 10 minutes to 60 minutes, then transferred to a medical grade tape, the microneedle array was stored in a dry environment.

Alternatively, a layer of surfactant was covered onto the surface of microporous cavity by a spray nozzle and compressed air, and then a hyaluronic acid solution was covered onto the surface of the microneedle mold with a surfactant coating by a dispenser connected with a syringe pump at a speed of 1 μL/min to 3 μl/min. After the coverage of hyaluronic acid solution on the mold, the microneedle was dried by a desiccant at room temperature (25° C.) for 10 minutes to 60 minutes, then transferred to a medical grade tape, the microneedle array was stored in a dry environment.

Thus, the present application developed a method for manufacturing a dissolving microneedle, which may solve the defects which existing in the method of present microneedle manufacturing. Because the manufacture method was not required to adopt a process of vacuum pumping or centrifugation, etc., it may be expanded to an industrial production scale, and the method needed not the repeat of the production process.

For example, during the manufacture of a microneedle, firstly the water was sprayed and filled into a microporous cavity of a microneedle mold and the air was removed, then a small amount of concentrated hyaluronic acid solution, for example, about 20 nL for a 280 micrometer microneedle, about 150 nL for a 500 micrometer microneedle, was directly dispensed into the top of each microporous cavity, to enable the hyaluronic acid solution to directly contact with water. Due to the natural hydrophobicity of the microneedle mold, the solution would not wet the surface of the microneedle mold and would ensure the hyaluronic acid solution to stay on the micropores of the mold that injected with water.

A concentration difference between the water in the microneedle mold and the hyaluronic acid solution on the top of the micropores of the mold was rapidly formed. The water gradually diffused into the upper layer, and the hyaluronic acid solution gradually diffused into the lower layer of microporous cavity, so that the concentration between them reached a balance. The diffusion efficiency of the concentrated hyaluronic acid and water could be monitored in a real-time manner during the drying process. In actual production, the concentration difference was formed instantaneously, because the methylene blue dye in the upper layer had been evenly distributed throughout the cavity of the microneedle mold in just one minute. With the water evaporation, the hyaluronic acid droplets placed on top of the micropores gradually enter the cavity of the microneedle mold, and at the late stage of the drying process, the dried hyaluronic acid may fill in the cavity of the microneedle mold adequately. Vacuum pumping or centrifugation was unnecessary during the diffusion process. The diffusion rate of active substances into the cavity of the microneedle mold was affected by ambient temperature and humidity. For a 50% hyaluronic acid solution (by weight or volume ratio), it took about 5 minutes to completely diffuse into the mold. Thus, it was very important to firstly spray and fill water into the cavity of the microneedle mold for enhancing the yield of microneedle, because an incomplete, stump-like microneedle structure would be formed if this step was omitted.

After dryness, the adhesive bottom tape was applied on the microneedle mold and further applied to the microneedle. The microneedle was pulled out of the mold by the use of adhesive tape to obtain a dissolving microneedle array arranged on the bottom substrate, which could be directly applied to the skin, and required no additional backing layer. All of these processes could be integrated into a fully automated continuous production line. The dissolving microneedle of which the active substance was accumulated on the needle head may be manufactured by use of the hyaluronic acid with low concentration or reduction of the volume of droplets. After final dryness, a small amount of dry solids were accumulated at the top of the mold cavity or forming a hollow microneedle, and the remaining space on the microneedle may be loaded with a second layer of substance.

Finally, it should be emphasized that the present application is not limited to the foregoing embodiments and the variations or modifications such as the changes in the shape of a microporous cavity in a microneedle mold, the changes in a polymer solution used in the microneedle, etc., shall be fall into the scope of protection of the claims of the present application. Therefore, various changes and modifications could be made herein by those skilled in the art. Any modifications, equivalent substitutions, improvements, etc. made without departing from the spirit and principles of this application shall fall into the scope of protection of the present application.

### Eleventh Embodiment

The present embodiment provided a device for manufacturing dissolving microneedle, comprising a microneedle mold and a high pressure spray device. The microneedle mold had a plurality of microporous cavities, and the high pressure spray device was configured to spray a polymer solution into the microporous cavity, and filled the polymer solution into each microporous cavity.

### INDUSTRIAL APPLICABILITY

In summary, the present application provides a method for manufacturing dissolving microneedle. The method is simple in process, high in manufacturing efficiency and low in production cost.

1. A method for manufacturing dissolving microneedle, comprising manufacturing a microneedle mold, said microneedle mold has a plurality of microporous cavities, wherein a polymer solution is sprayed into the microporous cavity by a high pressure spraying device to enable the polymer solution to fill into each of the microporous cavities; and the solidified microneedle are extracted from the microneedle mold after the dryness of polymer solution.

2. The method for manufacturing dissolving microneedle according to claim 1, wherein said polymer solution sprayed into the microporous cavity by the high pressure spray device comprises the atomized polymer solution sprayed into the microporous cavity by a high pressure atomizing device.

3. The method for manufacturing dissolving microneedle according to claim 2, wherein the particle size of said atomized polymer solution sprayed by the high pressure atomizing device ranges from 0.1 to 100 micrometers.

4. The method for manufacturing dissolving microneedle according to claim 1, wherein said polymer solution sprayed into the microporous cavity by the high pressure spray device comprises the droplets of the polymer solution sprayed to the microporous cavity by the high pressure spotting device.

5. The method for manufacturing dissolving microneedle according to claim 4, wherein the particle size of said droplets of the polymer solution sprayed by the high pressure spotting device ranges from 0.1 to 1500 micrometers.

6. The method for manufacturing dissolving microneedle according to claim 1, wherein said polymer solution is sprayed directly into the microporous cavity.

7. The method for manufacturing dissolving microneedle according to claim 1, wherein before said polymer solution is sprayed into said microporous cavity, water or an active drug solution is sprayed into said microporous cavity, and said polymer solution is sprayed to the water or the active drug solution, and when said polymer solution is uniformly mixed with the water or the active drug solution and dried, the microneedle are extracted.

8. The method for manufacturing dissolving microneedle according to claim 1, wherein before the polymer solution is sprayed to the microporous cavity, a surfactant is sprayed to the polymer solution, and the polymer solution is sprayed onto the surfactant, and after the polymer solution is uniformly mixed with the surfactant and dryness, the microneedle are extracted.

9. The method for manufacturing dissolving microneedle according to claim 8, wherein said surfactant is filled onto a surface of the microporous cavity, and there is a filling space formed in the microporous cavity that filled with said surfactant.

10. The method for manufacturing dissolving microneedle according to claim 9, wherein the polymer solution is a polymer mixed solution mixed with an active drug; the polymer mixed solution is sprayed into the filling space by the high pressure spray device.

11. The method for manufacturing dissolving microneedle according to claim 1, wherein the pressure value of the high pressure spray device spraying a polymer solution into the microporous cavity is less than 3.2 MPa.

12. The method for manufacturing dissolving microneedle according to claim 1, wherein after the polymer solution is sprayed into the microneedle mold by the high pressure spray device, the polymer solution is filled into each of the microporous cavities, and after the dryness of the polymer solution, the polymer solution merely adheres to the inner wall of the microporous cavity.

13. The method for manufacturing dissolving microneedle according to claim 1, wherein the polymer solution is sprayed to the microneedle mold by the high pressure spray device to ensure the polymer solution to adhere to the surface of the microporous cavity of the microneedle mold, thereby to form a hollow dissolving microneedle; the drug droplets to be loaded are sprayed into microneedle with a hollow structure by a spray nozzle of a high pressure spotting device, and the drug droplets are filled into microneedle to form a conical structure.

14. The method for manufacturing dissolving microneedle according to claim 1, wherein a sticky patch is pasted onto the surface of the microneedle, then the sticky patch is contacting with the surface of the dissolving microneedle by pressing; the dissolving microneedle pasted on the sticky patch is separated from the microneedle mold by lifting and pulling.

15. The method for manufacturing dissolving microneedle according to claim 1, wherein the polymer solution is a polymer solution mixed with a drug.

16. A device for manufacturing dissolving microneedle, comprising a microneedle mold and a high pressure spray device, wherein the microneedle mold has a plurality of microporous cavities and the high pressure spray device is configured to spray a polymer solution into the microporous cavity and to enable the polymer solution to be tilled in each of the microporous cavities.
